(19) United States
(12) Patent Application Publication
(10) Pub. No.: US 2009/0081789 A1
(43) Pub. Date: Mar. 26, 2009

(54) Activation of nuclear factor kappa B

(75) Inventors: Thomas E. Wagner, Greenville, SC (US); Gunter Schwamberger, Salzburg (AT); Xianzhong Yu, Mauldin, SC (US); Yanzhang Wei, Greer, SC (US)

Correspondence Address: FOLEY AND LARDNER LLP;SUITE 500, 3000 K STREET NW, WASHINGTON, DC, 20007, US

(73) Assignee: Greenville Hospital System

(21) Appl. No.: 12/149,086

(22) Filed: Apr. 25, 2008

Related U.S. Application Data
(60) Provisional application No. 60/935,817, filed on Aug. 31, 2007.

(51) Int. Cl. C12N 5/02

(52) U.S. Cl. 435/375

(57) ABSTRACT

The present invention describes a method for targeting a tumor cell comprising contacting the tumor cell with a composition comprising a macrophage and a factor that upregulates nuclear factor-kappa B (NFκB) activity.

### CROSS-REFERENCE TO RELATED PATENT APPLICATIONS

This application claims priority from U.S. Provisional Application 60/935,817, filed Aug. 31, 2007, incorporated herein by reference in its entirety.

### FIELD OF THE INVENTION

The present invention relates to targeted activation of nuclear factor-kappa B (NFκB) for anti-tumor therapy.

### BACKGROUND OF THE INVENTION

Nuclear factor-kappa B (NFκB) is a transcription factor that functions in regulating the immune response to infection by binding to a specific DNA sequence, GGGACTTTCC, within the intronic enhancer of the immunoglobulin kappa light chain in mature B-cells and plasma cells. NFκB is found in most cell types and acts as an intracellular transducer of external stimuli to activate a large number of genes in response to infections, inflammation and other stressful situations (Karin et al., Annu. Rev. Immunol. 18: 621-663, 2000). For instance, NFκB responds to and induces IL-2; NFκB induces TAP1 and MHC molecules, as well as inflammatory response-associated factors, e.g. IL-1, TNF-α and leukocyte adhesion molecules. As NFκB is a regulator of genes that control proliferation, differentiation and survival of lymphocytes, it is not surprising that activation of NFκB effects the oncogenesis of many lymphoid malignancies.

The activity of NFκB is tightly regulated by its interaction with the inhibitory proteins in the signaling pathways (Heissmeyer et al., Molecular and Cellular Biology 21: 1024-1035, 2001; and Nishikori, J. Clin. Exp. Hematopathol. 45: 15-24, 2005). In unstimulated cells, inhibitors of kappa B (IκB) bind to NFκB and mask the nuclear localization signals (NLS) of NFκB such that NFκB is sequestered in the cytoplasm in its inactive form. Ling et al. (Proc. Natl. Acad. Sci. USA, 95: 3792-3797, 1998) describe that NFκB-inducing kinase (NIK) is triggered by inflammatory cytokines, such as TNF and IL-1, to activate IκB kinase-α (IKK-α) by phosphorylating the serine at position 176 of IKK-α. Subsequently, the serine residues at positions 32 and 36 of IκB are phosphorylated by IKK-α, which results in ubiquitination and proteosome-mediated degradation of IκB. It follows that NFκB is freed from its binding to IκB and enters nucleus to regulate the expression of a number of genes. It has been demonstrated that a mutation of Ser-176 of IKK-α to Glu-176 (S176E) causes prolonged activation of NFκB.

NFκB is a known crucial mediator of macrophage inflammatory responses. In particular, NFκB mediates the cell attacking function of the macrophages. Activation of NFκB may have a negative impact, however, because it is responsible for the up-regulation of TNF-α, IL-1, interferons, etc., which can lead to patient death. Hence, it is presumed that in order to benefit from the up-regulation of gene delivery via macrophage by way of activation of NFκB, the downstream activation of TNF-α; IL-1, interferons and other proinflammatory mediators must be turned off to avoid any negative effect on the patient.

The inventors of the present application are the first to selectively activate NFκB in a targeted manner to achieve continuous, long term activation and specific tumor cell killing. This is accomplished with tumor targeted delivery of a factor that upregulates NFκB locally, and without activation of other signaling pathways.

### SUMMARY OF THE INVENTION

In the first aspect, the present invention provides a methodology for killing tumor cells. The method comprises (i) transfecting a macrophage by contacting the macrophage with a composition comprising (a) a nucleic acid component that can activate nuclear factor-kappa B via, for example, release from inhibition by IκB, (b) a lysosome evading component, and (c) a particle that can be phagocytosed; and (ii) contacting the tumor cells with the transfected macrophage from step (i). Components (a), (b), and (c) are collectively referred to herein as a particle conjugated virus. In one embodiment, the lysosome evading component is a non-replicative and/or non-infective, form of a virus or component of a virus. In another embodiment, the nucleic acid component can act as a lysosome evading component and therefore, a second additional lysosome evading component is optional. For example, the nucleic acid component can comprise a non-replicative or non-infectious form of a virus containing a nucleic acid sequence that encodes a protein that activates NFκB.

In some embodiments, the nucleic acid component may be DNA or RNA. In one embodiment, the nucleic acid may encode a protein or a RNAi construct. For instance, the nucleic acid may encode a protein that is associated with the NFκB signaling pathway and can activate NFκB, such as IKK-α with a mutation at position 176 from serine to glutamic acid. In yet another embodiment, the nucleic acid may be an siRNA construct for IκB. Furthermore, the nucleic acid component comprises a nucleic acid encoded in an expression vector containing a promoter, such as a hypoxia induced promoter, a promoter targeted by an immunosuppressive cytokine such as TGF-β, stress promoters, and other promoters that get upregulated selectively within a tumor tissue. Additional suitable promoters are those which can be activated by a drug or other signal when applied to the tumor tissue locally. For example, suitable promoters can be turned on locally in the tumor tissue by external means such as the radioinducible elements of the Egr-1 promoter (Kufe 2003) and the p21/WAF1/CIP1 promoter (Nenoi 2006) driven by focused gamma-irradiation, or the hsp70 promoter, which is driven by local heating, for instance.

According to the present invention, the particle to be phagocytosed is not limited by shape or material, and is one that approximates the size of the microbial structures that monocytic cells typically ingest. In one embodiment, the particle will be about 0.05 to about 5.0 μm, about 0.05 to about 2.5 μm, about 0.1 to about 2.5 μm, about 1.0 to about 2.5 μm, about 1.0 to about 2.0 μm, or about 1.0 to about 1.5 μm. The term “about” in this context refers to +/−0.1 μm. In one embodiment, the particle is a digestible particle from a natural source, such as a microbial particulate structure. For example, the particle that can be a phagocytosed is yeast cell wall particle, such as zymosan, or a beta glucan or a peptidoglycan from gram positive bacteria. Other suitable particles that can be phagocytosed, however, include agarose and inulin. In another embodiment, the particle to be phagocytosed is a particle that has a ferro-magnetic center covered by a polymer coat. Preferably, the ferro-magnetic particles are Dynabeads™ (Dynal Biotech), which are monodisperse polystyrene microspheres that are available in different sizes and are coated with various material. Other preferred ferro-magnetic particles are microbeads.

In some embodiments, the composition may further contain a nucleic acid protecting component, such as protamine, polyarginine, polylysine, histone, histone-like proteins, synthetic polycationic polymers or core protein of a retrovirus with the appropriate packaging sequence included in the RNA sequence.

The components may be attached to the particle by any means which allows for attachment. In one embodiment, the nucleic acid and the lysosome evading component are attached to the particle by antibody attachment. In another embodiment, the nucleic acid and the lysosome evading component are attached to the particle by interaction between (strept)avidin and biotin. In yet another embodiment, the nucleic acid serves as a multiple binding vehicle.

In another aspect, the invention provides a method for localized, targeted tumor killing. The method comprises delivering a composition comprising the particle conjugated virus of the present invention to a macrophage, transfecting a macrophage with the particle conjugated virus, and contacting a tumor with the transfected macrophages. The macrophages may be first transfected with a particle conjugated virus ex vivo and then reinfused into the patient, or the particle conjugated virus may be administered directly without prior contact with macrophages before administration.

In yet another embodiment, a particle conjugated virus is put in contact with a macrophage ex vivo and the supernatant following macrophage transfection is collected and administered for anti-tumor therapy. The supernatant can be concentrated and/or antitumor-active material purified and used for cancer treatment.

### BRIEF DESCRIPTION OF THE DRAWINGS

FIG. 1 is an adenovirus vector containing siRNA for Iκβ gene fused to green fluorescence protein (GFP).

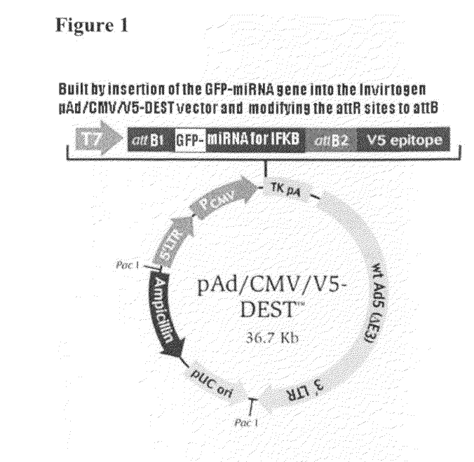

FIG. 2 depicts the stimulation of macrophage anti-tumor activity by adenovirus-mediated gene transfer. The anti-tumor activity is displayed as % cytotoxicity of YAC-1 tumor cells incubated with macrophages either unstimulated or stimulated with IFN-γ (control group). Bacterial lipopolysaccharide (LPS) serves as a positive control for induction of macrophage anti-tumor activity when applied together with IFN-γ. Before addition of the tumor cells, the macrophages were transfected with two different RNAi constructs for IκB, MB-Ad406 and MB-Ad407, respectively; or control Ad-MB-vectors lacking the RNAi constructs (MB-AdGFP).

The only difference between the 406 and 407 constructs is the sequences of the siRNA IκB. For construct 406, the top sequences are

5′TGCTGTTCAGAAGTGCCTCAGCAATTGTTTTGGCCACTGACTGACAAT
TGCTGGCACTTCTGAA 3′;

and the bottom:

5′CCTGTTCAGAAGTGCCAGCAATTGTCAGTCAGTGGCCAAAACAATTG
CTGAGGCACTTCTGAAC 3′.

For construct 407, the top sequences are

5′TGCTGTCAACAAGAGCGAAACCAGGTGTTTTGGCCACTGACTGACAC
CTGGTTGCTCTTGTTGA 3;

and the bottom:

5′CCTGTCAACAAGAGCAACCAGGTGTCAGTCAGTTGCCAAAACACCTG
GTTTCGCTCTTGTTGAC 3′.

FIG. 3 depicts nitric oxide (NO) production by the IFN-γ-stimulated or transfected macrophages. NO production is displayed as concentration of nitrite (NO₂) generated by macrophages either unstimulated or stimulated with IFN-γ (control group). Bacterial lipopolysaccharide (LPS) serves as a positive control when applied together with IFN-γ. Before addition of the tumor cells, the macrophages are transfected with two different RNAi constructs for IκB, MD-Ad406 and MB-Ad407, respectively; or control Ad-MB-vectors lacking the RNAi constructs (MB-AdGFP).

### DETAILED DESCRIPTION OF THE INVENTION

### Introduction

The present invention provides a methodology for killing tumor cells. The method comprises (i) contacting a macrophage with a composition comprising a nucleic acid component that comprises a factor that activates nuclear factor-kappa B, a particle to be phagocytosed by macrophages, and a lysosome evading component, and (ii) contacting a tumor cell with the macrophage transfected in (i).

Particle that can be Phagocytosed

The particle that can be phagocytosed is not limited by shape or material. In general, the particle can be of any shape, size or material that allows it to be phagocytosed by macrophages. The particle can be from a synthetic source or a natural source.

In one embodiment, the particle that can be phagocytosed has a ferro-magnetic center covered by a polymer coat. Preferred ferro-magnetic particles are Dynabeads™. (Dynal Biotech). Dynabeads™ are monodisperse polystyrene microspheres that are available in different sizes and are coated with various material. Other exemplary ferro-magnetic particles are microbeads and magnetic separation can be employed with the microbeads to separate free from bead-bound components during processing.

In another embodiment, the particle to be phagocytosed is one that is digestible and approximates the size of the microbial structures that monocytic cells typically ingest. A particularly preferred particle is a particle from sources, preferably of microbial origin, and most preferably a yeast cell wall particle. In one embodiment, the yeast cell wall particle is a zymosan particle. Zymosan (also referred to as Zymosan A) is commercially available from various companies such as. Sigma-Aldrich. For manufacturing purposes, slightly larger particles are preferred, because they are less likely to stick together, and so washing free from bound components is easier with the larger particle sizes. The zymosan particle size is typically about 2.0 μm.

A preferred size for the particle is one that approximates the size of microbial structures that macrophages typically ingest. In one embodiment, the particle will be about 0.05 to about 5.0 μm, about 0.05 to about 2.5 μm, about 0.1 to about 2.5 μm, about 1.0 to about 2.5 μm, about 1.0 to about 2.0 μm, or about 1.0 to about 1.5 μm. The term “about” in this context refers to +/−0.1 μm.

### Nucleic Acid Component

The particle of the present invention generally is attached to a nucleic acid component. The nucleic acid component comprises a nucleic acid that encodes a protein or siRNA that can activate NFκB. The nucleic acid component can be composed of DNA, RNA, both DNA and RNA, or dsRNA. The nucleic acid component can also comprise a vector which contains the nucleic acid, such as an adenovirus vector or an RNA virus that comprises dsRNA that inhibits expression of genes involved in the downregulation or decreases expression of NFκB. The component typically contains the signals necessary for translation and/or transcription (i.e., it can ultimately encode a protein or an RNA product) of the nucleic acid that can activate NFκB.

In one embodiment, the nucleic acid component comprises an RNAi construct that affects one or more factors associated with the NFκB signaling pathway. For example, the nucleic acid component comprises an RNAi construct that inactivates the expression of IκB. Since IκB, the inhibitor of NFκB, is inactivated, NFκB activity is up-regulated. Similarly, activators of IκB can also be inhibited by siRNA to ultimately increase NFκB activity.

In another embodiment, the nucleic acid component comprises a nucleic acid that encodes a protein that affects one or more factors associated with the NFκB signaling pathway. For example, the nucleic acid may encode a mutant IKK-α protein, where the serine at position 176 is replaced by glutamic acid. This mutant IKK-α is known to activate NFκB. In another embodiment, a protein upstream of IKK can be activated, such as IRAK4, and/or TAK1, by creating a constitutively phosphorylated mutant protein. Such a mutant protein can be made by, for example, substituting a serine residue for glutamic acid.

The skilled artisan immediately will comprehend which proteins can be encoded by the nucleic acid. Any suitable protein for use in the present invention will be one that ultimately leads to local activation of NFκB activity. The proteins will be expressed predominantly in the immediate vicinity of a tumor via the tumor-associated macrophage.

The nucleic acid component may also comprise a vector which contains the nucleic acid under the control of a promoter. Preferably, the promoter operably linked to the coding sequence is a hypoxia induced promoter. Because the tumor cells are normally hypoxic, a hypoxia induced promoter will assist in upregulation of NFκB activity locally at the target tissue, such as in a tumor region. Other exemplary promoters include a promoter targeted by an immunosuppressive cytokine such as TGF-β, stress promoters that can be activated by local irradiation or application of an inducer, and other promoters that get upregulated selectively within a tumor tissue. Additional suitable promoters are those which can be activated by a drug or other signal when applied to the tumor tissue locally.

Suitable promoters include Smad-complex responsive elements, heme oxidase 1 promoter, STAT6 responsive elements, radioinducible elements of the Egr-1 promoter, p21/WAF1/CIP1 promoter, or hsp70 promoter. In one embodiment, a suitable promoter for use in the present invention when targeting a tumor cell would be a hypoxia induced promoter, such as a hypoxia responsive element.

The vector may further comprise a selectable marker sequence, for instance for propagation in in vitro bacterial or cell culture systems. Preferred expression vectors comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 or cytomegalovirus (CMV) viral genome, for example, SV40 origin, early promoter, enhancer, splice, and polyadenylation sites may be used to provide the required nontranscribed genetic elements.

Specific initiation signals may also be required for efficient translation of inserted target gene coding sequences. These signals include the ATG initiation codon and adjacent sequences. In cases where a nucleic acid component includes its own initiation codon and adjacent sequences are inserted into the appropriate expression vector, no additional translation control signals may be needed. However, in cases where only a portion of an open reading frame (ORF) is used, exogenous translational control signals, including, perhaps, the ATG initiation codon, must be provided. Furthermore, the initiation codon must be in phase with the reading frame of the desired coding sequence to ensure translation of the entire target.

These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544 (1987)). Some appropriate expression vectors are described by Sambrook, et al., in Molecular Cloning: A Laboratory Manual, Second Edition, Cold Spring Harbor, N.Y. (1989), the disclosure of which is hereby incorporated by reference. If desired, to enhance expression and facilitate proper protein folding, the codon context and codon pairing of the sequence may be optimized, as explained by Hatfield et al., U.S. Pat. No. 5,082,767.

Exemplary vectors include pAd/CMV/V5-DEST (Invitrogen).

### Lysosome Evading Component

When a macrophage ingests a large antigen, a phagocytic vesicle (phagosome) which engulfs the antigen is formed. Next, a specialized lysosome contained in the macrophage fuses with the newly formed phagosome. Upon fusion, the phagocytized large antigen is exposed to several highly reactive molecules as well as a concentrated mixture of lysosomal hydrolases. These highly reactive molecules and lysosomal hydrolases digest the contents of the phagosome. Therefore, by attaching a lysosome evading component to the particle, the nucleic acid that is also attached to the particle escapes digestion by the materials in the lysosome and enters the cytoplasm of the macrophage intact. Prior systems failed to recognize the importance of this feature and, thus, obtained much lower levels of expression than the present invention. See Falo et al., WO 97/11605 (1997). It should be noted that the term “lysosome evading component” encompasses the fused lysosome/phagosome described above.

In addition to the nucleic acid component that can up-regulate NFκB activity, the composition of the present invention also comprises a lysosome evading component. The lysosome evading component and the nucleic acid component may be one in the same, or a separate component that is attached to the nucleic acid component. The role of the lysosome evading component is to assist the nucleic acid component in escaping the harsh environment of the lysosome.

Thus, the lysosome evading component is any component that is capable of evading or disrupting the lysosome. For example, the lysosome evading component can include proteins, carbohydrates, lipids, fatty acids, biomimetic polymers, microorganisms and combinations thereof. It is noted that the term “protein” encompasses a polymeric molecule comprising any number of amino acids. Therefore, a person of ordinary skill in the art would know that “protein” encompasses a peptide, which is understood generally to be a “short” protein. Preferred lysosome evading components include proteins, viruses or parts of viruses. The adenovirus penton protein, for example, is a well known complex that enables the virus to evade/disrupt the lysosome/phagosome. Thus, either the intact adenovirus or the isolated penton protein, or a portion thereof (see, for example, Bal et al., Eur J Biochem 267:6074-81 (2000)), can be utilized as the lysosome evading component. Fusogenic peptides derived from N-terminal sequences of the influenza virus hemagglutinin subunit HA-2 may also be used as the lysosome evading component (Wagner, et al., Proc. Natl. Acad. Sci. USA, 89:7934-7938, 1992).

Other preferred lysosome evading components include biomimetic polymers such as Poly (2-propyl acrylic acid) (PPAAc), which has been shown to enhance cell transfection efficiency due to enhancement of the endosomal release of a conjugate containing a plasmid of interest (see Lackey et al., Abstracts of Scientific Presentations: The Third Annual Meeting of the American Society of Gene Therapy, Abstract No. 33, May 31, 2000-Jun. 4, 2000, Denver, Colo.). Examples of other lysosome evading components envisioned by the present invention are discussed by Stayton, et al. J. Control Release, 1; 65(1-2):203-20, 2000.

### Nucleic Acid Protection Component

In addition to the components described above which are generally attached to the particle, either directly or via attachment to one another (e.g., a recombinant adenovirus encoding a nucleic acid), other components may also be attached to the particle or to a component that is attached to the particle. For example, a DNA protecting component may optionally be added to the particle containing compositions described above, especially where the nucleic acid component is not associated with a virus or a portion thereof. Generally, the DNA protecting component will not be attached directly to the particle. The nucleic acid protecting component includes any component that can protect the particle-bound DNA or RNA from digestion during brief exposure to lytic enzymes prior to or during lysosome disruption. Preferred nucleic acid protecting components include protamine, polyarginine, polylysine, histone, histone-like proteins, synthetic polycationic polymers and core protein of a retrovirus with the appropriate packaging sequence included in the RNA sequence.

In one embodiment of the present invention, the composition of the present invention comprises (i) a nucleic acid component that comprises a recombinant, optionally non-replicative and/or non-infective, virus or part of a virus, which contains a nucleic acid that encodes a protein that activates NFκB, or an siRNA that increases NFκB activity, and (ii) a particle to be phagocytized. The virus may be an RNA virus, like a retrovirus, or a DNA virus, like an adenovirus. In this embodiment, the virus itself preferably is capable of lysosome disruption. In other words, the virus is in both the nucleic acid and lysosome evading components. Alternatively, the virus may not be capable of lysosome disruption, and therefore, a separate lysosome evading component should be added. Preferred viruses include HIV, adenovirus, Sindbis virus, and hybrid and recombinant versions thereof, such as an HIV-adenovirus hybrid, which is essentially a recombinant adenovirus that has been engineered to express HIV antigens. Viruses can be attached to the particles directly, using conventional methods. See Hammond et al., Virology 254:37-49 (1999).

Since viral infection is not essential in the present invention for the nucleic acid component to reach the cytoplasm of the macrophage, the virus can also be replication/infection deficient. One method for producing a replication/infection deficient adenovirus envisioned by the instant invention is altering the virus fiber protein. For example, a virus in which the fiber protein is engineered by specific mutations to allow the fiber protein to bind to an antibody but not to its cognate cellular receptor can be used in the instant invention.

Another method for producing a replication/infection deficient virus envisioned by the present invention is intentionally causing denaturation of the viral component responsible for infectivity. In the case of adenovirus, for example, the fiber protein could be disrupted during the preparation of the virus; for HIV it might be the envelope (env) protein. A method for producing a replication/infection deficient retrovirus envisioned by the present invention entails removing the outer membranes of the retrovirus so that only the retrovirus core particle remains. If a replication/infection deficient virus prepared as described above is attached to the yeast cell wall particle, a RNA protecting component, as described above, may also be attached to the particle.

In some therapeutic embodiments, it is beneficial for the vector to stably integrate into the target cell chromosome. For example, one mode for achieving stable integration is through the use of an adenovirus hybrid. Such an adenovirus hybrid involves, for example, an adenoviral vector carrying retrovirus 5′ and 3′ long terminal repeat (LTR) sequences flanking the DNA component encoding a therapeutic or antigenic nucleic acid or protein and a retrovirus integrase gene (see Zheng, et al. Nature Biotechnology, 18:176-180, 2000). In other embodiments, transient expression is preferred and cytoplasmic viruses, like Sindbis virus, can be employed. In such cases, where no lysosome evading component is naturally present on the virus, one is added. In the case of Sindbis or other such viruses, it can be engineered to express all or part of the adenovirus penton protein for this purpose, for example.

### Method for Attaching the Components to the Particle to be Phagocytosed

Attachment of the components discussed above to the particle to be phagocytosed can be accomplished by any means. As set out above, the various “components” include a nucleic acid that can up-regulate NFκB activity, and a lysosome evading component, which may both be present in a virus. Preferred methods for attachment include antibody attachment, biotin-(strept)avidin interaction and chemical crosslinking. Vector particle conjugates may be prepared with chemically attached antibodies, (strept)avidin or other selective attachment sites.

Antibody attachment can occur via any antibody interaction. Antibodies include, but are not limited to polyclonal antibodies, monoclonal antibodies (mAbs), humanized or chimeric antibodies, single chain antibodies including single chain Fv (scFv) fragments, Fab fragments, F(ab′)₂ fragments, fragments produced by a Fab expression library, anti-idiotypic (anti-Id) antibodies, epitope-binding fragments, and humanized forms of any of the above.

In general, techniques for preparing polyclonal and monoclonal antibodies as well as hybridomas capable of producing the desired antibody are well known in the art (Campbell, A. M., Monoclonal Antibody Technology: Laboratory Techniques in Biochemistry and Molecular Biology, Elsevier Science Publishers, Amsterdam, The Netherlands (1984); St. Groth et al., J. Immunol. Methods 35:1-21 (1980); Kohler and Milstein, Nature 256:495-497 (1975)), the trioma technique, the human B-cell hybridoma technique (Kozbor et al., Immunology Today 4:72 (1983); Cole et al., in Monoclonal Antibodies and Cancer Therapy, Alan R. Liss, Inc. (1985), pp. 77-96).

One example of antibody attachment encompassed by the present invention involves a single antibody which is chemically affixed to the particle to be phagocytosed. The antibody is specific to the component to be attached to the particle. Alternatively, two antibodies can be used. In this case, one antibody, attached to the particle is specific for a second antibody and the second antibody is specific to the component attached to the particle. Thus, the component-specific antibody binds the component, and that antibody, in turn, is bound by the particle-bound antibody. For instance, a goat- or rabbit-anti-mouse antibody may be bound to the particle and a mouse monoclonal antibody used to bind the specific component.

In another example of antibody attachment, protein A or any similar molecule with an affinity for antibodies, is employed. In this example, the particles are coated with protein A which binds to an antibody, which in turn is bound to the component being attached to the particle.

Attachment via biotin-(strept)avidin interaction may be accomplished, for instance, by attaching avidin to the particle and attaching biotin to the component to be attached. Chemical crosslinking may be accomplished by conventional means known to the artisan.

Another attachment mechanism involves the nucleic acid serving as a multiple binding vehicle. Synthetic gripper protein nucleic acid (PNA) oligonucleotides are designed to specifically bind to different nucleic acid sequences. PNA is a polynucleic acid analog with a peptide backbone rather than a deoxyribosephosphate backbone. These can be attached directly to the particle to be phagocytosed or derivatized for convenient attachment, thereby providing a sequence-specific means of attaching nucleic acid. Each gripper oligonucleotide can be derivatized or attached to different ligands or molecules and designed to bind different nucleic acid sequences. It is believed that the PNA interacts with the DNA via Hoogsteen base pairing interactions and that a stable PNA-DNA-PNA triplex clamp is formed (Zelphati, et al. BioTechniques, 28:304-316, 2000).

Thus, in one embodiment, one gripper is employed to bind the nucleic acid component to the particle and another is used to bind the lysosome evading component to the nucleic acid component. Many such iterations are possible. For example, a “gripper” comprising biotin can be sequence specifically bound at one site to the nucleic acid. Attachment to a particle coated with avidin occurs via biotin-avidin interaction. At another site on the nucleic acid, another “gripper” with a lysosome/phagasome evading component can be sequence specifically bound. Optionally, a “gripper” with a DNA protecting component can be sequence specifically bound to the nucleic acid at yet another site. Exemplary gripper oligonucleotides have been previously described.

In the case of attaching viruses to the particle, this can also be accomplished by engineering the virus to express certain proteins on its surface. For instance, the HIV env protein might be replaced with the adenovirus penton protein, or a portion thereof. The recombinant virus then could be attached via an anti-penton antibody, with attachment to the particle mediated, for example, by another antibody or protein A. In this embodiment, the penton protein also would serve as a lysosome evading component.

### Therapeutic Methods

Both in vivo and ex vivo therapeutic methods involving the composition or the transfected macrophages of this invention are contemplated. As for in vivo methods, the particle conjugated virus is generally administered parenterally, usually intravenously, intramuscularly, subcutaneously or intradermally. It may be administered, e.g., by bolus injection or continuous infusion. In ex vivo methods, macrophages are transfected with the particle conjugated virus outside the body and then preferably reinfused administered to the patient. For both methods, IFN-γ may also be administered as part of a combination therapy.

Targeting gene expression to a tumor using the particle conjugated virus of the instant invention is effective for cancer treatment. One type of cancer treatment encompassed by the instant invention involves targeting a nucleic acid component that can upregulate NFκB activity within a tumor tissue. This is accomplished by delivery of a particle conjugated virus to a macrophage, which is then attracted to a tumor.

It is known that as tumors, primary tumors and metastases alike, grow beyond a few millimeters in diameter and become deficient in oxygen, they secrete signal proteins to elicit several required events for the tumor's survival. These events include the secretion of signals which induce angiogenesis. As a part of the mechanism of angiogenic induction, hypoxic tumors secrete a signaling chemokine protein with the function of attracting monocytes to the tumor. Monocytes attracted to the sites of growing tumors then become macrophages and assist in the induction of tumor angiogenesis. Thus, the nucleic acid component that comprises a nucleic acid that upregulates NFκB activity is preferably under the control of a hypoxia induced promoter, although the other promoters described herein are also suitable. The macrophages transfected with the particle conjugated virus are then attracted to the sites of tumor development and deliver the nucleic acid component selectively to the tumor.

As provided above, interferon (IFN)-γ works as a strong enhancer and can be used in combination therapy with the present invention. Thus, either an IFN-γ gene with a suitable promoter can be used to produce IFN-γ in an autocrine way, or alternatively IFN-γ targeted genes may be induced directly via expression of altered STAT1 transcription factors, resembling the phosphorylated (active) form of STAT1. In addition, NF-IL6 may also enhance macrophage antitumor activity. And with regard to silencing, TNF-α is a suitable candidate. But TNF-α expression may also be useful for tumor destruction if produced locally.

The composition of this invention may be formulated for parenteral administration by, for example, local application (direct injection or microsurgery techniques), intramuscular or subcutaneous injection or intravenous injection for ex vivo applications (see above).

Formulations for injection may be presented in unit dosage form, e.g., in ampules or in multi-dose containers, optionally with an added preservative. The composition of this invention may take such forms as suspensions, solutions or emulsions in oily or aqueous vehicles, and may contain formulatory agents such as suspending, stabilizing and/or dispersing agents. The composition may also be formulated using a pharmaceutically acceptable excipient. Such excipients are well known in the art, but typically will be a physiologically tolerable aqueous solution. Physiologically tolerable solutions are those which are essentially non-toxic. Preferred excipients will either be inert or enhancing.

The following non-limiting examples are given by way of illustration only and are not to be considered limitations of this invention. There are many apparent variations within the scope of this invention.

### EXAMPLE 1

This example demonstrates the construction of siRNA for IκB. (Equal amounts of top and bottom strand miR oligos were annealed to generate double stranded oligos. The ds oligos were then ligated into linearized pcDNA6.2-GW/EmGFP-miR and transformed into One Shot TOP10 competent cells. Transformants were picked and plasmid DNA sequenced for confirmation of insertion of the miR ds oligo in the vector. The new vector was named pcDNA6.2-GW/EmGFP-miR IkB.

The newly generated pcDNA6.2-GW/EmGFP-miR IkB vector was linearized by Sac I digestion and purified. A BP recombination reaction was performed between the linearized vector and the donor vector pDONR221. 1 ul of the BP reaction was used to transform the TOP10 competent cells and correct transformants were selected by restriction enzyme digestion of the plasmid DNA. The plasmids at this step were named entry clones.

The correct entry clone was then used together with a destination vector pAd/CMV/V5-DEST in a LP recombination reaction. 2 ul of the LR recombination reaction mixture was used to transform the TOP10 competent cells and correct transformants were selected based on their resistance to ampicilin and sensitivity to chloramphenicol. Then plasmid DNA was prepared from those transformants and gel electrophoresis was performed to confirm the size of the final vector construction named pAd-EmGFP-miR IkB. Finally, the pAd-EmGFP-miR IkB plasmid DNA was transfected into a mammalian cell line to confirm the express of the EmGFP, which in turn confirm the existence of the miR IkB following EmGFP.)

The sequence of mouse siRNA IκB are as follows:

Top strand:
5′ TGCTGTCAACAAGAGCGAAACCAGGTGTTTTGGCCACTGACTGA
CACCTGGTTGCTCTTGTTGA 3′
Bottom strand:
5′ CCTGTCAACAAGAGCAACCAGGTGTCAGTCAGTGGCCAA
AACACCTGGTTTCGCTCTTGTTGAC 3′

### EXAMPLE 2

This example demonstrates stimulation of macrophage anti-tumor activity by adenovirus-mediated gene transfer.

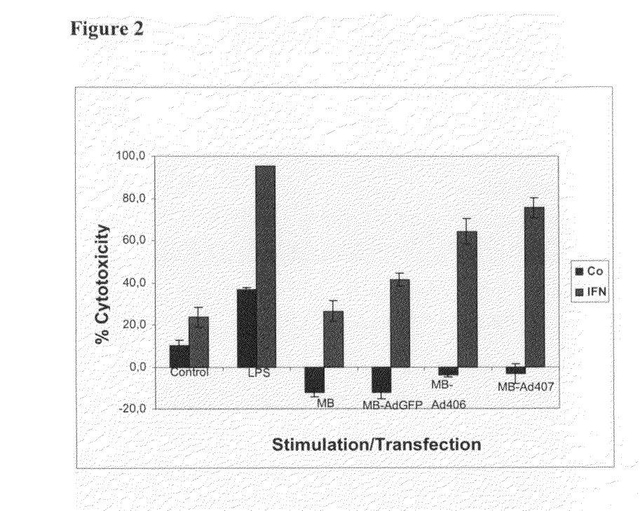

Thioglycollate elicted mouse peritoneal macrophages were transfected with Ad-MB-vectors at a ratio of approximately 4 magnetic beads (equivalent to about 40 Ad-particles) per macrophage for 16 hours, either with or without additional stimulation with interferon (IFN)-γ. Thereafter, culture medium was replaced by fresh medium without stimulants and YAC-1 mouse lymphoma cells added at an effector to target ratio of 10:1. After 48 hours, the number of remaining tumor cells was determined by measurement of alkaline phosphatase activity of the YAC-1 tumor cells, and results displayed as % cytotoxicity as compared to the control group of YAC-1 cells incubated without macrophages. Bacterial lipopolysaccharide (LPS) served as a positive control for induction of macrophage anti-tumor activity when applied together with IFN-γ. The results show enhanced tumor cytotoxic activity after transfection with two different RNAi constructs for IκB (MB-Ad406 and MB-Ad407), whereas magnetic beads alone (MB) or control Ad-MB-vectors lacking the RNAi constructs (MB-AdGFP) caused no or only modest enhancement of macrophage tumor cytotoxic activity (FIG. 2).

### EXAMPLE 3

This example demonstrates that enhanced cytotoxicity caused by Ad-MB vectors of the present invention is not due to enhanced NO radical production.

Parallel to determination of tumor cytotoxicity, production of NO-radicals by stimulated or transfected macrophages was determined via spectrophotometric assay (Griess reaction) of accumulated nitrite in macrophage culture supernatants at the end of the cytotoxicity assay.

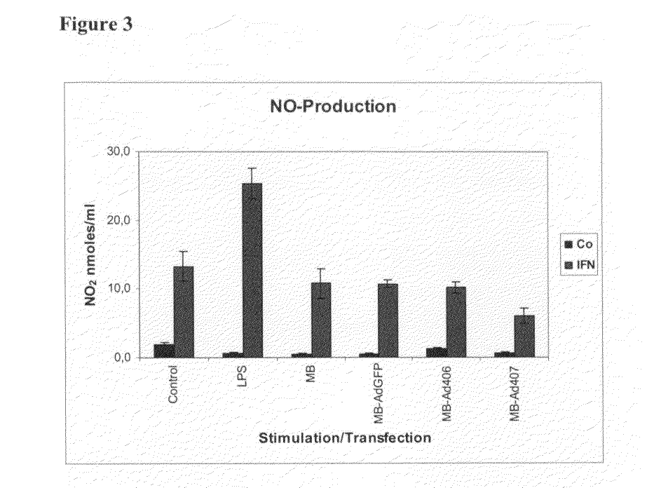

In contrast to cytotoxicity, no enhancement of NO production compared to stimulation with IFN-γ was observed for macrophages transfected with Ad-MB-vectors (FIG. 3), whereas LPS stimulation in conjunction with IFN-γ caused a marked increase in NO production. This result suggests that the enhanced cytotoxicity caused by Ad-MB transfection is not due to NO radicals. In addition, this may also indicate a more selective effect of Ad-MB transfection with IκB silencing constructs on macrophage anti-tumor functions as compared to stimulation with microbial products.

What is claimed is:

1. A method for killing tumor cells, comprising:
(i) contacting a macrophage with a composition comprising (a) a nucleic acid component that comprises a nucleic acid that upregulates nuclear factor-kappa B activity, (b) a lysosome evading component, and (c) a particle that can be phagocytosed; and
(ii) contacting the tumor cells with the macrophage obtained in (i).

2. The method of claim 1, wherein the nucleic acid component comprises DNA or RNA.

3. The method of claim 1, wherein the nucleic acid component comprises an expression vector.

4. The method of claim 3, wherein the expression vector contains a hypoxia induced promoter.

5. The method of claim 1, wherein the contacting step occurs ex vivo.

6. The method of claim 1, wherein the nucleic acid component comprises siRNA.

7. The method of claim 6, wherein the component comprises siRNA for IκB.

8. The method of claim 1, wherein the component comprises an RNAi construct.

9. The method of claim 1, wherein the lysosome evading component is a non-infectious virus or a non-infectious component of a virus.

10. The method of claim 9, wherein the virus is adenovirus.

11. The method of claim 9, wherein the virus is non-replicative.

12. The method of claim 1, wherein the lysosome evading component is a biomimetic polymer.

13. The method of claim 1, wherein the particle has a size between about 0.05 μm to about 5.0 μm.

14. The method of claim 1, wherein the particle has a size between about 1.0 μm to about 2.5 μm.

15. The method of claim 13, wherein the particle is a magnetic bead.

16. The method of claim 1, wherein the composition further comprises a nucleic acid protecting component.

17. The method of claim 16, wherein the protecting component is selected from the group consisting of protamine, polyarginine, polylysine, histone, histone-like proteins, synthetic polycationic polymers and a core particle of a retrovirus with the appropriate packaging sequence included in the RNA sequence.

18. The method of claim 1, wherein the nucleic acid component and the lysosome evading component are attached to the particle by antibody attachment.

19. The method of claim 1, wherein the nucleic acid component and the lysosome evading component are attached to the particle by interaction between (strept)avidin and biotin.

20. The method of claim 13, wherein the particle is a digestible particle from a microbial source.

21. The method of claim 9, wherein the lysosome evading component is the adenovirus penton protein.

22. The method of claim 20, wherein the particle is a yeast cell wall particle.

23. A composition comprising (a) a nucleic acid component that comprises a nucleic acid that upregulates the expression of nuclear factor-kappa B, (b) a lysosome evading component, and (c) a particle that can be phagocytosed.

24. The composition of claim 23, wherein the nucleic acid component comprises siRNA for IκB.


SEQUENCE LISTING (7 sequences)

SEQ ID NO: 1 - DNA, 10 bp, Artificial Sequence
  Description of Artificial Sequence Synthetic oligonucleotide
gggactttcc

SEQ ID NO: 2 - DNA, 64 bp, Artificial Sequence
  Description of Artificial Sequence Synthetic oligonucleotide
tgctgttcagaagtgcctcagcaattgttttggccactgactgacaattgctggcacttctgaa

SEQ ID NO: 3 - DNA, 64 bp, Artificial Sequence
  Description of Artificial Sequence Synthetic oligonucleotide
cctgttcagaagtgccagcaattgtcagtcagtggccaaaacaattgctgaggcacttctgaac

SEQ ID NO: 4 - DNA, 64 bp, Artificial Sequence
  Description of Artificial Sequence Synthetic oligonucleotide
tgctgtcaacaagagcgaaaccaggtgttttggccactgactgacacctggttgctcttgttga

SEQ ID NO: 5 - DNA, 64 bp, Artificial Sequence
  Description of Artificial Sequence Synthetic oligonucleotide
cctgtcaacaagagcaaccaggtgtcagtcagttgccaaaacacctggtttcgctcttgttgac

SEQ ID NO: 6 - DNA, 64 bp, Mus sp.; identical to SEQ ID NO: 4

SEQ ID NO: 7 - DNA, 64 bp, Mus sp.
cctgtcaacaagagcaaccaggtgtcagtcagtggccaaaacacctggtttcgctcttgttgac